(19) United States
(12) Patent Application Publication
(10) Pub. No.: US 2012/0059228 A1
(43) Pub. Date: Mar. 8, 2012

(54) METHODS FOR DETECTING ENDOMETRIOSIS

(75) Inventor: Ozgul Muneyyirci-Delale, Sands Point, NY (US)

(73) Assignee: THE RESEARCH FOUNDATION OF STATE UNIVERSITY OF NEW YORK, Albany, NY (US)

(21) Appl. No.: 13/256,770

(22) PCT Filed: Mar. 16, 2010
(86) PCT No.: PCT/US10/27395

§ 371 Date: Nov. 21, 2011

Related U.S. Application Data
(60) Provisional application No. 61/160,486, filed on Mar. 16, 2009.

(51) Int. Cl. A61B 5/00

(52) U.S. Cl. 600/300

(57) ABSTRACT

The method of the present invention is directed towards detecting endometriosis in a mammalian subject. This method comprises the following steps; taking a sample from the mammal, measuring the level of sphingomyelin (SM) or phosphatidylcholine (PC), or combinations thereof, in the sample, comparing the measured levels of SM or PC, or combinations thereof, to predetermined levels, and diagnosing the mammal based on the comparison. This method may also comprise the following steps: taking a sample from the mammal, measuring the level of F11 Receptor (F11R) in the sample, comparing the measured level of F11R to a predetermined level, and diagnosing the mammal based on the comparison.

### FIELD OF THE INVENTION

The invention relates generally to the field of the detection of endometriosis in a subject. More specifically, the present invention is directed to a method and kit to measure the level of Sphingomyelin (SM), phosphatidylcholine (PC), both SM and PC or F11R alone in a sample and comparing the measured levels to a predetermined level.

### BACKGROUND OF THE INVENTION

Endometriosis is a common gynecologic disease affecting 14 to 17% of reproductive age women and more than 40% of women with infertility. Although 90% of all cycling women experience retrograde menstruation, studies have shown that only 14-17% of these women develop endometriosis. Endometriosis is defined as the presence of viable endometrial tissue (glands and stroma) outside of the uterine cavity.

Several hypotheses have attempted to explain the development of endometriosis. Most theories of the pathogenesis of this disease involve some retrograde flow of endometrium with subsequent implantation. The process of implantation requires secretion of growth factors that allows neovascularization.

Endometriosis is a pelvic inflammatory process with altered function of immune-related cells and increased number of activated macrophages in the peritoneal environment that secrete various local products, such as growth factors and cytokines. The elevation of cytokines and other factors in the peritoneal fluid is accompanied by the elevation of similar factors, such as CRP, SAA, TNF-alpha, MCP-1, IL-6, IL-8 and CCR1, in the peripheral blood of patients with endometriosis. Endometriosis is known to be an inflammatory process associated with altered function of immunity-related cells and increased number of activated peritoneal macrophages and their secreted inflammatory mediators such as cytokines, growth factors, and angiogenic factors.

It has been hypothesized that the increased presence of lipid peroxidation products in the peritoneal fluid of endometriosis subjects may, at least partly, account for the recruitment of leukocytes, the increase in macrophage activation, the secretion of monocyte-macrophage-derived cytokines, and the endometrial growth-promoting activity associated with endometriosis.

At present, there are no reliable markers of endometriosis. Currently, the “gold” standard for the diagnosis of endometriosis is surgical creating a need for accurate laboratory diagnostic markers for endometriosis. Immunological markers have gained importance with the accumulating evidence regarding the immunological changes that occur during the evolution of the disease.

Identification of principal cellular and molecular factors of angiogenesis and fibrosis are also needed in order to develop new therapeutic strategies of endometriosis. There has been extensive research into the mechanisms of the disease and discovery of several potential diagnostic markers. Possible markers include interleukin-6, tumor necrosis factor-α, soluble Intercellular adhesion molecule-1 (sICAM-1), E selectin, Hepatocyte growth factor (HGF), Tissue Factor (TF), Vascular endothelial growth factor, Platelet-derived growth factor, Macrophage migration inhibitory factor, Early growth response (EGR)-1 gene, P450 aromatase, Placental Protein 14 (PP14), Macrophage chemotactic protein-1, Cytokeratins, Hormone receptors Interferon-g, Autoantibodies, Leptin, and CA-125.

The role of cell adhesion molecules (CAM) in inflammation is well documented. Recent work points to the involvement of adhesion molecules and growth factors in the initiation and regulation of endometriotic lesions on the microenvironment level.

F11Receptor (F11R) is a known. CAM and is a member of the immunoglobulin superfamily. It is a human ortholog of a murine CAM found in endothelial cells also called Junctional Adhesion Molecule-1 (JAM-1). Recent studies have revealed that F11R is a molecule critical for platelet secretion and aggregation and platelet adhesion to a cytokine-inflamed endothelial surface in vitro and that it may be an important mediator of the effects of inflammation.

What is desired is a simple diagnostic test which can determine a diagnosis for endometriosis. To determine a diagnosis through a diagnostic test, the presence of inflammation related to endometriosis is determined by the levels of SM and/or PC. Another method to determine a diagnosis through a diagnostic test is determined by the level of F11R. Further, what is desired is a kit that can be used to detect the presence of inflammation related to endometriosis as determined by SM and/or PC levels and determine the levels of F11R in a sample.

Embodiments of the present application provide a method and kit that addresses the above and other issues.

### SUMMARY OF THE INVENTION

The method of the present invention is directed towards detecting endometriosis in a mammalian subject. The method comprises taking a sample from a mammal, measuring the level of sphingomyelin (SM) or phosphatidylcholine (PC), or combinations thereof, in the sample, comparing the measured levels of SM or PC, or combinations thereof, to predetermined levels, and diagnosing a mammal based on the comparison. The method further comprises taking a sample from the mammal, measuring the level of F11 Receptor (F11R) in the sample, comparing the measured level of F11R to a predetermined level, and diagnosing the mammal based on the comparison.

### BRIEF DESCRIPTION OF THE FIGURES

The present invention will be better understood by reference to the following drawings of which:

FIG. 1 is a graphical representation of the measurement of SM and PC in a group of subjects;

FIG. 2 is a graphical representation of the measurement of F11R levels in a group of subjects;

FIG. 3 is a graphical representation of the measurement of SM levels in a group of subjects over time; and

FIG. 4 is a graphical representation of the measurement of F11R levels in a group of subjects over time.

### DETAILED DESCRIPTION OF THE INVENTION

The method of the present invention is a method for detecting endometriosis in a mammalian subject. The detection of decreased blood concentrations of certain markers associated with inflammation and atherosclerosis can be employed with the methods of the invention to assess the presence or absence of endometriosis in a relatively simple, cost-effective test. The improvements over the prior art are, inter alia, the performance of a non-surgical procedure which can determine accurate results quickly and inexpensively for a correct diagnosis of endometriosis. The method of the present invention may also be used to monitor the progress of treatment following a surgical procedure or administration of a medication. The kit of the present invention is a kit which contains the materials needed to collect sample material and determine a diagnosis based on the levels measured in the sample material.

In connection with the present invention, initially, a sample is taken from the mammalian subject. The mammalian subject may be, for example, a human, a cow, a horse, a pig, a dog or a cat. The sample can be taken or collected and may be composed of blood, tears, urine, saliva, peritoneal fluid or any other lipoprotein-containing material. Most preferably, the sample is blood. The sample can be taken or collected in any acceptable manner. A hypodermic needle may also be used to take or collect the sample. The volume of the sample can be any acceptable volume, for example, about 5 mL. The sample may be taken at any time. The preferred time to take the sample is during the early follicular phase of the female subject's menstrual cycle. The taking or collection of the present invention does not require the subject to fast prior to the taking of the sample.

In one embodiment, once the sample is taken, it is measured to determine the level of Sphingomyelin (SM), phosphatidylcholine (PC) or both. In another embodiment, once the sample is taken, it is measured to determine the level of F11R.

In one embodiment, a sample is taken which contains lipoproteins and two major phospholipids, SM and PC to detect the presence or absence of endometriosis. To measure the levels of SM and PC, an assay or enzymatic measurement is used. The level of SM, PC or both in the sample may be determined by any suitable assay or enzymatic measurement that accurately determines the corresponding levels of SM, PC or both. In one embodiment, the concentration of SM, PC or both can be determined by lipid extraction, thin layer chromatography, and phosphate determination on separated SM or PC spots. In one embodiment, the level of SM, PC or both can be determined by the following method which incorporates, for example, an enzyme-linked immunosorbent assay (ELISA). Any other suitable assay which can measure levels of SM, PC or both may also be used, including an immunofluorescence assay, an immunohistochemistry assay or a Western blot assay.

In another embodiment, the level of SM, PC or both can be determined by the following method. Initially the sample is incubated with bacterial sphingomyelinase (for SM measurement) or bacterial phospholipase D (for PC measurement), choline is generated which is used to generate hydrogen peroxide. Hydrogen peroxide is used together with DAOS (N-Ethyl-N-(2-hydroxy-3-sulfopropyl)-3,5-dimethoxyaniline, sodium salt), 4-aminoantipyrine and peroxidase as a catalyst, to generate a blue to purple dye, with an optimal absorption at 595 nm. The linear range for the SM measurement is about 0.5 to about 5 μg and the linear range for PC is about 2.5 to about 20 μg. To ensure the sample is within this range, serial dilutions can be created. A further description of this process is described in US 2009-0148877 A1, which is incorporated herein by reference.

In another embodiment, the level of F11R in the sample is measured to detect the presence or absence of endometriosis. To measure the level of F11R, an assay or enzymatic measurement is used. The level of F11R in the sample may be determined by any suitable assay that accurately determines the corresponding level of F11R in the sample.

In one embodiment, the level of F11R can be determined by the following method which incorporates, for example, an enzyme-linked immunosorbent assay (ELISA). Any other suitable assay which can measure levels of F11R may also be used, including an immunofluorescence assay, an immunohistochemistry assay or a Western blot assay. Initially a sample is added to wells of a microplate and is coated with a monoclonal antibody to F11R (M.Ab.F11R) overnight. The wells of the microplate are then blocked with phosphate-buffered saline-1% bovine serum albumin followed by a two hour incubation at 37° C. with 100 μL standard solutions of F11R protein followed by a two hour incubation at 37° C. with a detection biotinylated anti-human junctional adhesion molecule-1 antibody at a concentration of 200 ng/mL. Diluted streptavidin horseradish peroxidase (1:400) is applied and incubated for 30 minutes at room temperature with 100 μL of substrate solution and 50 μL of stop solution. Optical density measurements are then taken to determine F11R concentration.

Once the level of SM, PC, both or F11R in a sample is determined, the measured levels are compared to predetermined levels of SM, PC, both or F11R. Predetermined levels of SM, PC, both or F11R are gathered through statistically significant measurements of SM, PC and F11R levels in subjects without endometriosis. The predetermined level of SM is between about 50 mg/dL and about 150 mg/dL. More specifically, the predetermined level of SM may be between about 50 mg/dL and about 100 mg/dL. Even further, the predetermined level of SM may be between about 50 mg/dL and about 65 mg/dL. The predetermined level of PC is between about 200 mg/dL and about 400 mg/dL. More specifically, the predetermined level of PC may be between about 210 mg/dL and about 320 mg/dL. Even further, the predetermined level of PC may be between about 220 mg/dL, and about 270 mg/dL. The predetermined level of F11R is between about 60 pg/dL and about 500 pg/dL. More specifically, the predetermined level of F11R may be between about 80 pg/dL and about 250 pg/dL. Even further, the predetermined level of F11r may be between about 90 pg/dL and about 130 pg/dL. The measured level in the sample is compared to the predetermined levels and a diagnosis is made based on the comparison.

In one embodiment, a diagnosis of the subject having endometriosis is given if a measured SM level is about 20% lower than the predetermined SM level. For a positive diagnosis of the subject having endometriosis, the difference between the measured SM level and predetermined level can be about 20%, which, at the least, includes 16%, 17%, 18%, 19%, 21%, 22%, 23% and 24%.

In another embodiment, a diagnosis of the subject having endometriosis is given if a measured PC level is about 23% lower than the predetermined PC level. For a positive diagnosis of the subject having endometriosis, the difference between the measured PC level and predetermined level can be about 23%, which, at the least, includes 19%, 20%, 21%, 22%, 24%, 25%, 26% and 27%.

In another embodiment, a diagnosis of the subject having endometriosis is given if a measured SM level is about 20% lower than the predetermined SM level and a measured PC level is about 23% lower than the predetermined PC level. For a positive diagnosis of the subject having endometriosis, the difference between the measured SM level and predetermined level can be about 20%, which, at the least, includes 16%, 17%, 18%, 19%, 21%, 22%, 23% and 24% and the difference between the measured PC level and predetermined level can be about 23%, which, at the least, includes 19%, 20%, 21%, 22%, 24%, 25%, 26% and 27%.

In a further embodiment, a diagnosis of the subject having endometriosis is given if a measured F11R level is about 80% lower than the predetermined F11R level. For a positive diagnosis of the subject having endometriosis, the difference between the measured F11R level and predetermined level can be about 80%, which, at the least, includes 76%, 77%, 78%, 79%, 81%, 82%, 83% and 84%.

The method of the present invention also includes a method to monitor the progress of treatment following a surgical procedure or administration of a medication.

The method of the present invention may be used to detect the levels of SM or F11R after a subject has undergone a surgical procedure to remove endometriotic cells for a period of time. The method may be used to detect the levels of SM or F11R on a scheduled basis to determine if the surgical procedure causes an increase in the levels of SM or F11R. An increase in the levels of SM or F11R would indicate that the surgical procedure is successful in removing the endometriotic cells and that the subject's levels of SM or F11R were returning to a normal or control level.

The method of the present invention may be used to detect the levels of SM or F11R alter a subject has been administered a medication for a period of time. The medication or medications which could be administered to a subject with endometriosis could be any GnRH agonist including, but not limited to, buserelin, goserelin, leuprorelin, leuprolide, naferelin, triptorelin and/or any progestin including, but not limited to, norethynodrel, norethindrone acetate, norethindrone, norgestimate, norgestrel, levonorgestrel, medroxyprogesterone, desogestrel, etonogestrel and drospirenone. The method may be used to detect the levels of SM or F11R on a scheduled basis to determine if the administration causes an increase in the levels of SM or F11R. An increase in the levels of SM or F11R would indicate that the medication is successful in controlling the proliferation of endometriotic cells and that the subject's levels of SM or F11R were returning to a normal or control level. Example 3 below further describes detection of the levels of SM or F11R after a subject has been administered a medication for a period of time.

The present invention also includes a kit for diagnosing endometriosis in a mammalian subject. The kit includes a sampler which takes and collects a sample from the mammalian subject. In one embodiment, the sampler can be a hypodermic needle. The kit also includes an assay capable of determining the level of SM or F11R in the sample. All reagents, materials and components needed to perform the assay can also be included in the kit. Any assay capable of measuring SM or F11R in the sample can be included in the kit. An example of some assays which can be included in the kit are an enzyme-linked are an ELISA, an immunofluorescence assay, an immunohistochemistry assay and a Western blot assay. Also included in the kit is a recordation of the predetermined levels of SM or F11R. This recordation can be used by a practitioner to compare the measured level of SM or F11R in the sample and correlate the measured level to the predetermined levels.

The described embodiments of the present invention are intended to be illustrative rather than restrictive, and are not intended to represent every embodiment of the present invention. Various modifications and variations can be made without departing from the spirit or scope of the invention as set forth in the following claims both literally and in equivalents recognized in law.

### EXAMPLES

In a method for detecting endometriosis, several subjects were examined. In the endometriosis group, 40 women with surgical diagnosis of endometriosis and symptomatic for endometriosis (pelvic pain, dysemenorrhea and/or dyspareunia) had blood samples drawn in the early follicular phase of their menstrual cycle. In a control group, 12 normal cycling women with confirmed ovulation using Luteinizing Hormone (LH) surge and Basal Body Temperature (BBT) had blood samples drawn at a similar stage in the menstrual cycle (early follicular phase) as the group with endometriosis did. The mean age of the control group was 31±2 years, with the control group having a mean BMI of 24±1. The mean age of the endometriosis group was 35±1 years, with the endometriosis group having a mean BMI of 26±1. The samples were then measured for SM, PC and F11R levels.

### Example 1

The samples were measured for levels of Sphingomyelin (SM) and phosphatidylcholine (PC). SM was measured in a Plasma SM Assay with enzymatic measurement of plasma levels being done in a four step process:

1) bacterial SMase hydrolyzed SM to phosphorylcholine and n-acylsphingosine;

2) alkaline phosphatase generated choline from phosphorylcholine;

3) choline was used to generate hydrogen peroxide in a reaction catalyzed by choline oxidase; and

4) hydrogen peroxide was used together with DAOS, 4-aminoantipyrine, and peroxidase, as a catalyst, to generate a blue dye with an optimal absorption at 595 nm.

PC was measured with enzymatic measurement of plasma levels being done in a three step process:

1) bacterial phospholipase D (specific for PC, no reaction with SM) hydrolyzed PC to choline and phosphatidic acid;

2) choline was used to generate hydrogen peroxide in a reaction catalyzed by choline oxidase; and

3) hydrogen peroxide was used together with DAOS, 4-aminoantipyrine, and peroxidase, as a catalyst, to generate a blue dye with an optimal absorption at 595 nm.

The results of the measurement of SM and PC were recorded. The data was expressed as the mean±standard error. The student t-test was used to compare the two study groups (p≦0.05 considered as significant).

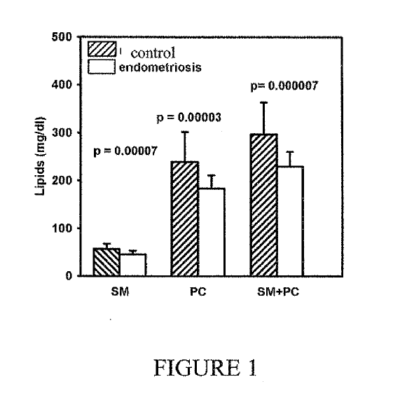

Upon collection of all results, SM levels were significantly higher in the control group compared to the endometriosis group (57.3 mg/dL±2.9 vs. 45.95 mg/dL±1.20: p<0.001). PC levels also were significantly higher in the control group than the endometriosis group (239.8 mg/dL±17.2 vs. 183.9 mg/dL±4.3: p<0.001). These results can be seen in FIG. 1.

SM and PC levels were lower in endometriosis patients when compared to normal cycling women in the control group.

### Example 2

The samples were separately measured for levels of F11R. A sensitive enzyme-linked immunosorbent assay (ELISA) was used to determine F11R levels. Recombinant proteins, antibodies (AB) & reagents for the ELISA were obtained from R&D Systems, Inc. (Minneapolis, Minn.) & HyCult biotechnology (Uden, Netherlands). The standard curve using concentrations of recombinant F11R is 0.1 pg/mL to 200 ng/mL. Serial dilutions may be used to ensure the sample is within this range of F11R level.

Monoclonal antibody F11R (M.Ab.F11R) was provided at 100 μL/mL and coated overnight at 4° C. onto microplate wells. After coating, the microplate wells were blocked with phosphate-buffered saline-1% bovine serum albumin (PBS-1% BSA) followed by a 2 hour incubation at 37° C. with 100 μL standard solutions of F11R protein followed by a 2 hour incubation at 37° C. with a detection biotinylated anti-human JAM-1 Ab (200 ng/mL). Diluted streptavidin-horseradish peroxidase (1:400) was applied and incubated for 30 minutes at room temperature with 100 μL of substrate solution and 50 μL of stop solution. Optical density measurements were then taken at 450 mm (microplate reader from Bio-Rad Laboratories-model 650, Hercules, Calif.).

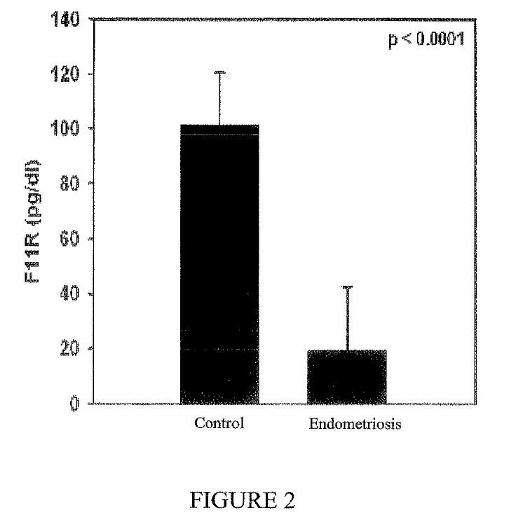

Upon measurement of all samples, data was recorded and expressed as the mean±standard error. Student t-test was used to compare the two study groups (P≦0.05 considered as significant). ANOVA analysis (SPSS Version 15, © 2006) was performed to look for correlations between F11R concentrations with age and BMI. F11R levels were compared between the control group and the endometriosis group. The mean F11R level in the control group was 101.1 pg/dL±19.7. The mean F11R level in the endometriosis group was 18.9 pg/dL±4.1. These results can be seen in FIG. 2. Serum (sF11R) did not correlate with age in the control group, however it inversely correlated with age in the endometriosis group, (p=0.05). sF11R did not correlate with BMI in either group.

F11R levels in women with symptomatic endometriosis are lower than those levels in normal healthy women.

### Example 3

Women with symptomatic endometriosis were randomized in a double-blind placebo controlled study comparing the effects of a dosage of 11.25 mg of leuprolide and a 5 mg dosage of norethindrone acetate. For subjects being given a dosage of leuprolide, an 11.25 mg dosage was injected at zero and 12 weeks. For subjects being given a dosage of norethindrone acetate, a 5 mg dosage was ingested daily for the 24 week period. F11R levels were assayed by ELISA which included recombinant proteins, antibodies (AB) & reagents for the ELISA were obtained from R&D Systems, Inc. (Minneapolis, Minn.) & BD Biosciences (San Diego, Calif.). Levels of SM were measured in a Plasma SM Assay as described in Example 1 above. The results are expressed as the mean±the standard error.

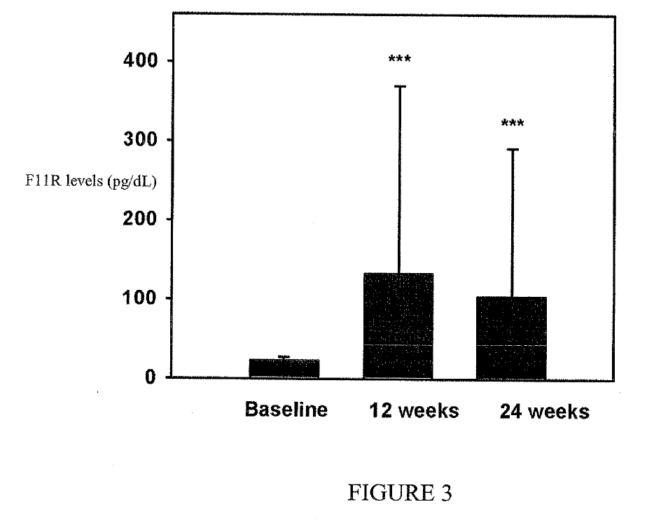

F11R values at baseline, prior to administration of the medication, week 12 and week 24 were 22.7+4.23, 133.0+236.5 and 103.9+187.2 pg/dL respectively. The F11R levels were significantly higher at week 12 (P=0.005) and week 24 (p=0.008) when compared with baseline levels. There was no significant differences between week 12 and week 24 F11R levels (p=0.16). The combined results of the leuprolide and norethindrone acetate treatments can be seen in FIG. 3.

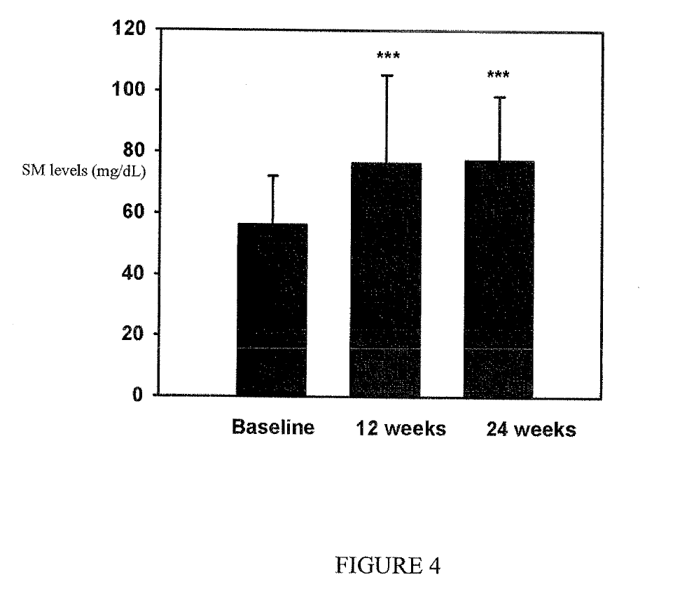

The SM levels at baseline, prior to administration of the medication, 12 weeks and 24 weeks after treatment were 56.34±15.82, 76.44±28.94 and 77.46±21.00 mg/dl respectively. The week 12 and 24 SM levels were significantly higher than baseline levels, the p value being <0.0001 for both. There were no significant differences in SM levels of week 12 and week 24 (p=0.76). The combined results of the leuprolide and norethindrone acetate treatments can be seen in FIG. 4.

What is claimed is:

1. A method of detecting endometriosis in a mammal comprising:
taking a sample from the mammal;
measuring the level of sphingomyelin (SM) or phosphatidylcholine (PC), or combinations thereof, in the sample;
comparing the measured levels of SM or PC, or combinations thereof, to predetermined levels; and
diagnosing the mammal based on the comparison.

2. The method of claim 1 wherein the sample is taken during the early follicular phase of the mammal's menstrual cycle.

3. The method of claim 1 wherein a diagnosis of having endometriosis is given if a measured SM level is about 20% lower than the predetermined level.

4. The method of claim 1 wherein a diagnosis of having endometriosis is given if a measured PC level is about 23% lower than the predetermined level.

5. The method of claim wherein a diagnosis of having endometriosis is given if a measured SM level is about 20% lower than the predetermined level and a measured PC level is about 23% lower than the predetermined level.

6. The method of claim 1 wherein the mammal is a human.

7. The method of claim 1 wherein the sample is selected from the group consisting of blood, tears, urine, peritoneal fluid and saliva.

8. The method of claim 7 wherein the sample is blood.

9. The method of claim 1 wherein the measured level of SM, PC, or combinations thereof, is measured by assay.

10. The method of claim 9 wherein only the SM level is measured by assay.

11. The method of claim 9 wherein only the PC level is measured by assay.

12. The method of claim 9 wherein both the SM level and the PC level is measured by assay.

13. The method of claim 1 wherein the measured level of SM, PC, or combinations thereof, is measured by enzymatic measurement.

14. The method of claim 13 wherein only the SM level is measured by enzymatic measurement.

15. The method of claim 13 wherein only the PC level is measured by enzymatic measurement.

16. The method of claim 13 wherein both the SM level and the PC level is measured by enzymatic measurement.

17. A kit for diagnosing endometriosis in a mammal comprising: a sampler, an assay capable of determining the level of SM, PC, or combinations thereof in a sample, components for performing the assay, and a predetermined level of SM, PC, or combinations thereof for comparison.

18. The kit of claim 17 wherein the assay is selected from the group consisting of an enzyme-linked immunosorbent assay (ELISA), immunofluorescence, immunohistochemistry and a Western blot.

19. The kit of claim 17 wherein the sampler is a hypodermic needle.

20. A method of detecting endometriosis in a mammal comprising:
taking a sample from the mammal;
measuring the level of F11 Receptor (F11R) in the sample;
comparing the measured level of F11R to a predetermined level; and
diagnosing the mammal based on the comparison.

21. The method of claim 21 wherein the sample is taken during the early follicular phase of the mammal's menstrual cycle.

22. The method of claim 21 wherein a diagnosis of having endometriosis is given if a measured F11R level is about 80% lower than the predetermined level.

23. The method of claim 21 wherein the mammal is a human.

24. The method of claim 21 wherein the sample is selected from the group consisting of blood, tears, urine, peritoneal fluid and saliva.

25. The method of claim 25 wherein the sample is blood.

26. The method of claim 21 wherein the measured level of F11R is measured by assay.

27. A kit for diagnosing endometriosis in a mammal comprising: a sampler, an assay capable of determining F11R levels in a sample, and a predetermined level of F11R for comparison.

28. The kit of claim 28 wherein the assay is selected from the group consisting of an enzyme-linked immunosorbent assay (ELBA), immunofluorescence, immunohistochemistry and a Western blot.

29. The kit of claim 28 wherein the sampler is a hypodermic needle.
